(19) United States
(12) Patent Application Publication
(10) Pub. No.: US 2021/0299161 A1
(43) Pub. Date: Sep. 30, 2021

(54) THERAPUETIC HYDROGEN GAS GENERATION AND DELIVERY

(71) Applicant: H2 Therapeutics, Blackfoot, ID (US)

(72) Inventors: Jared Barrott, Chubbuck, ID (US); Kerry Casperson, Blackfoot, ID (US); Taft Barrott, Starr, ID (US); Hugo Rayo, Pocatello, ID (US); William Cannon, Pocatello, ID (US)

(21) Appl. No.: 17/199,236

(22) Filed: Mar. 11, 2021

Related U.S. Application Data
(60) Provisional application No. 62/993,978, filed on Mar. 24, 2020.

(51) Int. Cl. A61K 33/00; A61M 16/00; A61M 16/20; A61M 16/06; A61M 16/10; A61M 16/12; A61M 5/14

(52) U.S. Cl. CPC A61K 33/00; A61M 16/024; A61M 16/208; A61M 16/0666; A61M 2205/3327; A61M 16/12; A61M 5/14; A61M 2202/02; A61M 16/105

(57) ABSTRACT

For hydrogen generation and delivery, a hydrogen source provides hydrogen. A delivery tube delivers the hydrogen from the hydrogen source for inhalation. A hydrogen sensor measures hydrogen concentration in the delivery tube. A controller adjusts the hydrogen concentration in the delivery tube to a concentration threshold.

### CROSS-REFERENCE TO RELATED APPLICATIONS

This application claims priority to U.S. Provisional Patent Application No. 62/993,978 entitled “H2Tx hydrogen generator with CPAP devices” and filed on Mar. 24, 2020 for Jared J. Barrot, which is incorporated herein by reference.

### FIELD

The subject matter disclosed herein relates to therapeutic hydrogen gas generation and delivery.

### BACKGROUND

### DESCRIPTION OF THE RELATED ART

Small amounts of inhaled hydrogen gas are therapeutic.

### BRIEF SUMMARY

An apparatus for hydrogen generation and delivery is disclosed. The apparatus includes a hydrogen source, a deliver tube, a hydrogen sensor, and a controller. The hydrogen source provides hydrogen. The delivery tube delivers the hydrogen from the hydrogen source for inhalation. The hydrogen sensor measures hydrogen concentration in the delivery tube. The controller adjusts the hydrogen concentration in the delivery tube to a concentration threshold. A system and method also perform the functions of the apparatus.

### BRIEF DESCRIPTION OF THE DRAWINGS

A more particular description of the embodiments briefly described above will be rendered by reference to specific embodiments that are illustrated in the appended drawings. Understanding that these drawings depict only some embodiments and are not therefore to be considered to be limiting of scope, the embodiments will be described and explained with additional specificity and detail through the use of the accompanying drawings, in which:

FIG. 1 is a schematic block diagram illustrating one embodiment of a hydrogen system;

FIG. 2 is a schematic block diagram illustrating one embodiment of hydrogen data;

FIG. 3 is a perspective drawing illustrating one embodiment of a hydrogen system;

FIG. 4 is a perspective drawing illustrating one embodiment of an electrolysis chamber;

FIG. 5A is a perspective drawing illustrating one embodiment of a delivery adapter;

FIG. 5B is a perspective drawing illustrating one alternate embodiment of a delivery adapter;

FIG. 6 is a schematic block diagram illustrating one embodiment of a controller; and

FIG. 7 is a schematic flow chart diagram illustrating one embodiment of a hydrogen gas generation and delivery method.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

The term “and/or” indicates embodiments of one or more of the listed elements, with “A and/or B” indicating embodiments of element A alone, element B alone, or elements A and B taken together.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

The description of elements in each figure may refer to elements of proceeding figures. Like numbers refer to like elements in all figures, including alternate embodiments of like elements.

The cells in our body are constantly experiencing oxidative stress and generating free radicals. Free radicals are linked to some of the most pervasive and debilitating diseases in humans, including inflammatory diseases, cardiovascular diseases, neurological disorders, aging and cancer. For decades, humans have tried to combat oxidative stress with antioxidants in foods or in supplements. An emerging therapy to treat conditions linked to oxidative stress is to consume hydrogen water. Water consisting of one oxygen bonded to two hydrogens can be combined with extra hydrogen gas to generate hydrogen water. This solution provides the excess hydrogen atoms to bind and nullify the negative effects of free radicals. More recent studies involving hydrogen water have looked at the benefits of vaporizing hydrogen water and inhaling it. Due to the small size of hydrogen and its neutral charge, hydrogen molecules can penetrate any tissue and cellular compartment including the mitochondria and nucleus. In fact, the data demonstrates that hydrogen can reach maximum concentration levels within 5 minutes, including tissues like the brain. This emphasizes the utility of dosing hydrogen water over traditional antioxidants that might not penetrate all tissues and cells very well. Scientific studies have already demonstrated that hydrogen water consumption can facilitate more successful liver transplants, slow the progression of cancer and neurodegenerative disease. And as recently as last month, scientists and clinicians are using hydrogen therapy to treat patients with lung injury as a result of COVID-19 infections. Four clinical trials have already been initiated. Despite the strong evidence to support the beneficial effects of hydrogen water, the acceptance of this therapy in more disease settings has been hampered by some studies showing no benefits. This could be due to the fact that how much hydrogen gas to add to water and how much to consume is not standardized. Also, hydrogen water has a very short shelf life if not handled properly or consumed shortly after opening a bottle. Importantly to note, the Food and Drug Administration (FDA) has classified hydrogen water as generally recognized as safe (GRAS), meaning humans can consume it without the risk of side effects.

Safety of hydrogen therapy

In May of 2014, the Office of Food Additive Safety, a branch of the Food and Drug Administration (FDA), filed a letter for the use of molecular hydrogen (H2) as a beverage additive and was given the generally recognized as safe (GRAS) status. Under the GRAS status, H2 is exempt from the premarket approval required by the Federal Food, Drug and Cosmetic Act. After completing the investigation into the safety of H2, the scientists concluded that hydrogen-enriched beverages are suitable for persons of all ages and medical conditions, and there is currently no evidence in the scientific literature that hydrogen-enriched beverages should not be consumed by any segment of the population.

H2 saturation in water is 1.8 parts per million (PPM)

Half-live or diffusion kinetics of H2 is about 2 hours.

Maximum body absorption is reached within 15 minutes.

H2 distribution equals the total volume of water in the body.

Over 300 peer-reviewed scientific studies have been published regarding the safety and benefits of hydrogen therapy. Below are selected references to support the different administrations and disease settings in which hydrogen has been used.

Human Studies

Shin M H, Park R, Nojima H, Kim H C, Kim Y K, Chung J H. Atomic hydrogen surrounded by water molecules, H(H2O)m, modulates basal and UV-induced gene expressions in human skin in vivo. PLoS One.

Yoritaka A, Takanashi M, Hirayama M, Nakahara T, Ohta S, Hattori N. Pilot study of H(2) therapy in Parkinson's disease: a randomized double-blind placebo-controlled trial. Mov Disord. 2013;28(6):836-9.

Ono H, Nishijima Y, Adachi N, Tachibana S, Chitoku S, Mukaihara S, et al. Improved brain Mill indices in the acute brain stem infarct sites treated with hydroxyl radical scavengers, Edaravone and hydrogen, as compared to Edaravone alone. A non-controlled study. Med Gas Res. 2011;1(1):12.

Nagatani K, Nawashiro H, Takeuchi S, Tomura S, Otani N, Osada H, et al. Safety of intravenous administration of hydrogen-enriched fluid in patients with acute cerebral ischemia: initial clinical studies. Med Gas Res. 2013;3(1):13.

Xia C, Liu W, Zeng D, Zhu L, Sun X, Sun X. Effect of hydrogen-rich water on oxidative stress, liver function, and viral load in patients with chronic hepatitis B. Clin Transl Sci. 2013;6(5):372-5.

Matsumoto S, Ueda T, Kakizaki H. Effect of supplementation with hydrogen-rich water in patients with interstitial cystitis/painful bladder syndrome. Urology. 2013;81(2):226-30.

Kato S, Saitoh Y, Iwai K, Miwa N. Hydrogen-rich electrolyzed warm water represses wrinkle formation against UVA ray together with type-I collagen production and oxidative-stress diminishment in fibroblasts and cell-injury prevention in keratinocytes. J Photochem Photobiol B. 2012;106:24-33.

Ono H, Nishijima Y, Adachi N, Sakamoto M, Kudo Y, Nakazawa J, et al. Hydrogen(H2) treatment for acute erythymatous skin diseases. A report of 4 patients with safety data and a non-controlled feasibility study with H2 concentration measurement on two volunteers. Med Gas Res. 2012;2(1):14.

Ishibashi T, Ichikawa M, Sato B, Shibata S, Hara Y, Naritomi Y, et al. Improvement of psoriasis-associated arthritis and skin lesions by treatment with molecular hydrogen: A report of three cases. Mol Med Rep. 2015;12(2):2757-64.

Li Q, Kato S, Matsuoka D, Tanaka H, Miwa N. Hydrogen water intake via tube-feeding for patients with pressure ulcer and its reconstructive effects on normal human skin cells in vitro. Med Gas Res. 2013;3(1):20.

Ishibashi T, Sato B, Rikitake M, Seo T, Kurokawa R, Hara Y, et al. Consumption of water containing a high concentration of molecular hydrogen reduces oxidative stress and disease activity in patients with rheumatoid arthritis: an open-label pilot study. Med Gas Res. 2012;2(1):27.

Ishibashi T, Sato B, Shibata S, Sakai T, Hara Y, Naritomi Y, et al. Therapeutic efficacy of infused molecular hydrogen in saline on rheumatoid arthritis: a randomized, double-blind, placebo-controlled pilot study. Int Immunopharmacol. 2014;21(2):468-73.

Ito M, Ibi T, Sahashi K, Ichihara M, Ito M, Ohno K. Open-label trial and randomized, double-blind, placebo-controlled, crossover trial of hydrogen-enriched water for mitochondrial and inflammatory myopathies. Med Gas Res. 2011;1(1):24.

Aoki K, Nakao A, Adachi T, Matsui Y, Miyakawa S. Pilot study: Effects of drinking hydrogen-rich water on muscle fatigue caused by acute exercise in elite athletes. Med Gas Res. 2012;2(1):12.

Ostojic S M, Vukomanovic B, Calleja-Gonzalez J, Hoffman J R. Effectiveness of oral and topical hydrogen for sports-related soft tissue injuries. Postgrad Med. 2014; 126(5): 187-95.

Sakai T, Sato B, Hara K, Hara Y, Naritomi Y, Koyanagi S, et al. Consumption of water containing over 3.5 mg of dissolved hydrogen could improve vascular endothelial function. Vasc Health Risk Manag. 2014;10:591-7.

Kajiyama S, Hasegawa G, Asano M, Hosoda H, Fukui M, Nakamura N, et al. Supplementation of hydrogen-rich water improves lipid and glucose metabolism in patients with type 2 diabetes or impaired glucose tolerance. Nutr Res. 2008;28(3):137-43.

Song G, Li M, Sang H, Zhang L, Li X, Yao S, et al. Hydrogen-rich water decreases serum LDL-cholesterol levels and improves HDL function in patients with potential metabolic syndrome. J Lipid Res. 2013;54(7):1884-93.

Song G, Lin Q, Zhao H, Liu M, Ye F, Sun Y, et al. Hydrogen activates ATP-binding cassette transporter A1-dependent efflux ex vivo and improves high-density lipoprotein function in patients with hypercholesterolemia: a double-blinded, randomized and placebo-controlled trial. J Clin Endocrinol Metab. 2015;100:2724-33.

Nakao A, Toyoda Y, Sharma P, Evans M, Guthrie N. Effectiveness of hydrogen rich water on antioxidant status of subjects with potential metabolic syndrome-an open label pilot study. J Clin Biochem Nutr. 2010;46(2):140-9.

Nakayama M, Kabayama S, Nakano H, Zhu W J, Terawaki H, Nakayama K, et al. Biological effects of electrolyzed water in hemodialysis. Nephron Clin Pract. 2009;112(1):c9-15.

Nakayama M, Nakano H, Hamada H, Itami N, Nakazawa R, Ito S. A novel bioactive haemodialysis system using dissolved dihydrogen (H2) produced by water electrolysis: a clinical trial. Nephrol Dial Transplant. 2010;25(9):3026-33.

Terawaki H, Zhu W J, Matsuyama Y, Terada T, Takahashi Y, Sakurai K, et al. Effect of a hydrogen (H2)-enriched solution on the albumin redox of hemodialysis patients. Hemodial Int. 2014;18(2):459-66.

Terawaki H, Hayashi Y, Zhu W J, Matsuyama Y, Terada T, Kabayama S, et al. Transperitoneal administration of dissolved hydrogen for peritoneal dialysis patients: a novel approach to suppress oxidative stress in the peritoneal cavity. Med Gas Res. 2013;3(1):14.

Animal Studies

Ohsawa I, Ishikawa M, Takahashi K, Watanabe M, Nishimaki K, Yamagata K, et al. Hydrogen acts as a therapeutic antioxidant by selectively reducing cytotoxic oxygen radicals. Nat Med. 2007;13(6):688-94.

Gharib B, Hanna S, Abdallahi O M, Lepidi H, Gardette B, De Reggi M. Anti-inflammatory properties of molecular hydrogen: investigation on parasite-induced liver inflammation. C R Acad Sci III. 2001;324(8):719-24.

Ito M, Hirayama M, Yamai K, Goto S, Ito M, Ichihara M, et al. Drinking hydrogen water and intermittent hydrogen gas exposure, but not lactulose or continuous hydrogen gas exposure, prevent 6-hydorxydopamine-induced Parkinson's disease in rats. Med Gas Res. 2012;2(1):15.

Sobue S, Yamai K, Ito M, Ohno K, Ito M, Iwamoto T, et al. Simultaneous oral and inhalational intake of molecular hydrogen additively suppresses signaling pathways in rodents. Mol Cell Biochem. 2015;403(1-2):231-41.

Qiu X, Li H, Tang H, Jin Y, Li W, Sun Y, et al. Hydrogen inhalation ameliorates lipopolysaccharide-induced acute lung injury in mice. Int Immunopharmacol. 2011;11(12):2130-7.

Manaenko A, Lekic T, Ma Q, Zhang J H, Tang J. Hydrogen inhalation ameliorated mast cell-mediated brain injury after intracerebral hemorrhage in mice. Crit Care Med. 2013;41(5):1266-75.

Sun Y, Shuang F, Chen D M, Zhou R B. Treatment of hydrogen molecule abates oxidative stress and alleviates bone loss induced by modeled microgravity in rats. Osteoporos Int. 2013;24(3):969-78.

Hong Y, Shao A, Wang J, Chen S, Wu H, McBride D W, et al. Neuroprotective effect of hydrogen-rich saline against neurologic damage and apoptosis in early brain injury following subarachnoid hemorrhage: possible role of the Akt/GSK3beta signaling pathway. PLoS One. 2014;9(4):e96212.

Abe T, Li X K, Yazawa K, Hatayama N, Xie L, Sato B, et al. Hydrogen-rich University of Wisconsin solution attenuates renal cold ischemia-reperfusion injury. Transplantation. 2012;94(1):14-21.

Song G, Tian H, Qin S, Sun X, Yao S, Zong C, et al. Hydrogen decreases athero-susceptibility in apolipoprotein B-containing lipoproteins and aorta of apolipoprotein E knockout mice. Atherosclerosis. 2012;221(1):55-65.

Zhang C B, Tang Y C, Xu X J, Guo S X, Wang H Z. Hydrogen gas inhalation protects against liver ischemia/reperfusion injury by activating the NF-kappaB signaling pathway. Exp Ther Med. 2015;9(6):2114-20.

Kohama K, Yamashita H, Aoyama-Ishikawa M, Takahashi T, Billiar T R, Nishimura T, et al. Hydrogen inhalation protects against acute lung injury induced by hemorrhagic shock and resuscitation. Surgery. 2015;158(2):399-407.

Kawamura T, Wakabayashi N, Shigemura N, Huang C S, Masutani K, Tanaka Y, et al. Hydrogen gas reduces hyperoxic lung injury via the Nrf2 pathway in vivo. Am J Physiol Lung Cell Mol Physiol. 2013;304(10):L646-56.

Li Y, Xie K, Chen H, Wang G, Yu Y. Hydrogen gas inhibits high-mobility group box 1 release in septic mice by upregulation of heme oxygenase 1. J Surg Res. 2015;196(1):136-48.

Wei R, Zhang R, Xie Y, Shen L, Chen F. Hydrogen Suppresses Hypoxia/Reoxygenation-Induced Cell Death in Hippocampal Neurons Through Reducing Oxidative Stress. Cell Physiol Biochem. 2015;36(2):585-98.

Fujita K, Seike T, Yutsudo N, Ohno M, Yamada H, Yamaguchi H, et al. Hydrogen in drinking water reduces dopaminergic neuronal loss in the 1-methyl-4-phenyl-1,2,3,6-tetrahydropyridine mouse model of Parkinson's disease. PLoS One. 2009;4(9):e7247.

Chen C H, Manaenko A, Zhan Y, Liu W W, Ostrowki R P, Tang J, et al. Hydrogen gas reduced acute hyperglycemia-enhanced hemorrhagic transformation in a focal ischemia rat model. Neuroscience. 2010;169(1):402-14.

Hugyecz M, Mracsko E, Hertelendy P, Farkas E, Domoki F, Bari F. Hydrogen supplemented air inhalation reduces changes of prooxidant enzyme and gap junction protein levels after transient global cerebral ischemia in the rat hippocampus. Brain Res. 2011;1404:31-8.

Li J, Wang C, Zhang J H, Cai J M, Cao Y P, Sun X J. Hydrogen-rich saline improves memory function in a rat model of amyloid-beta-induced Alzheimer's disease by reduction of oxidative stress. Brain Res. 2010;1328:152-61.

Xiao X, Cai J, Xu J, Wang R, Cai J, Liu Y, et al. Protective effects of hydrogen saline on diabetic retinopathy in a streptozotocin-induced diabetic rat model. J Ocul Pharmacol Ther. 2012;28(1):76-82.

Qu J, Li X, Wang J, Mi W, Xie K, Qiu J. Inhalation of hydrogen gas attenuates cisplatin-induced ototoxicity via reducing oxidative stress. Int J Pediatr Otorhinolaryngol. 2012;76(1):111-5.

Nakashima-Kamimura N, Mori T, Ohsawa I, Asoh S, Ohta S. Molecular hydrogen alleviates nephrotoxicity induced by an anti-cancer drug cisplatin without compromising anti-tumor activity in mice. Cancer Chemother Pharmacol. 2009; 64(4):753-61.

Amitani H, Asakawa A, Cheng K, Amitani M, Kaimoto K, Nakano M, et al. Hydrogen improves glycemic control in typel diabetic animal model by promoting glucose uptake into skeletal muscle. PLoS One. 2013;8(1):e53913.

Hayashida K, Sano M, Kamimura N, Yokota T, Suzuki M, Ohta S, et al. Hydrogen inhalation during normoxic resuscitation improves neurological outcome in a rat model of cardiac arrest independently of targeted temperature management. Circulation. 2014;130(24):2173-80.

Our bodies are constantly generating chemical reactions that can result in free radicals. Free radicals are molecules in our bodies that destructively interact with DNA and proteins within cells causing damage to our tissues termed oxidative stress. Everybody experiences oxidative stress every day of our lives. Antioxidants are the antidote to free radicals and help maintain good cellular health. A simple, yet powerful antioxidant is molecular hydrogen. It is the smallest element and can bind to each other to form H2 gas. When molecular hydrogen comes into contact with free radicals, they neutralize the free radical, and one of the most common reaction outcomes is the production of H2O or water. Hydrogen therapy appears to be a panacea for a number of human illnesses that are result of chronic oxidative stress. Hydrogen therapy has been studied in the context of a number of diseases and has shown positive benefits. There are several methods to introduce hydrogen therapy to humans including infusing beverages with hydrogen gas, dissolving hydrogen tablets in beverages, intravenous injections, or inhalation of the gas. However, there are inherent problems with each route of administration.

Hydration-Infused Beverages

Whether it is by direct infusion using hydrogen gas or dissolving tablets, the max capacity of water under normal pressure is 1.8 parts per million (PPM). Under higher bottling pressure, higher concentrations can be achieved, but the storage and retention of hydrogen in normal bottles or even metal-lined bottles is not very long. Once exposed to air, the half-life of hydrogen in solution is about 2 hours and most manufacturers recommend drinking the beverage within 30 minutes for the full effect. Hydrogen dissolved tablets produce even less concentrated beverages (0.2 PPM). In conclusion, hydrogen-infused beverages have short shelf lives and do not produce the most potent dose of hydrogen therapy.

Intravenous Injections

Intravenous injections are only performed in Japan. This is the most invasive procedure to introduce hydrogen therapy to a consumer. It does provide the fastest time to reach maximum distribution; however, due to the small nature of molecular hydrogen, it has a very fast and ubiquitous distribution by oral and inhalation administration, thereby negating the benefits of performing intravenous dosing.

Inhalation Delivery

Breathing hydrogen gas is an acceptable route of administration and allows for the highest exposure to hydrogen, thus maximizing the benefits of hydrogen therapy against oxidative stress. The air we breathe contains minimal traces of hydrogen gas (0.00005 PPM). Hydrogen gas becomes combustible in the range of 4-70%, thus the maximum recommended dose is 4% or 40,000 PPM H2 gas. Saturation is reached within 15 minutes for a normal individual. Studies have shown that intermittent exposure to inhalation hydrogen therapy achieves greater health benefits compared to continuous administration.

Molecular Properties of Hydrogen Gas

Hydrogen gas is the smallest of all the elements and can easily diffuse throughout the body. In every tissue throughout the body there are ongoing chemical reactions. This can be a result of the air we breathe, the food we ingest, or even the sunlight that we are exposed to. A common byproduct of these reactions is the production of free radicals. The accumulation of these free radicals is also called oxidative stress because they are typically an oxygen molecule with a lone pair electron that is highly reactive with DNA and proteins within our cells. This causes damage to important cellular functions and cause death of the cells or change the cells' behavior resulting in different diseases. Hydrogen gas exists in the form H2 (2 atoms of hydrogen bound together). In the presence of free radicals, the hydrogen will split apart and bind to the lone pair electron of the reactive oxygen. The most common free radical is a hydroxyl free radical (.OH), thus when a hydrogen binds to this free radical the outcome is H2O or water. Hydrogen is a very effective neutralizer of free radicals, thereby maintaining excellent cellular health.

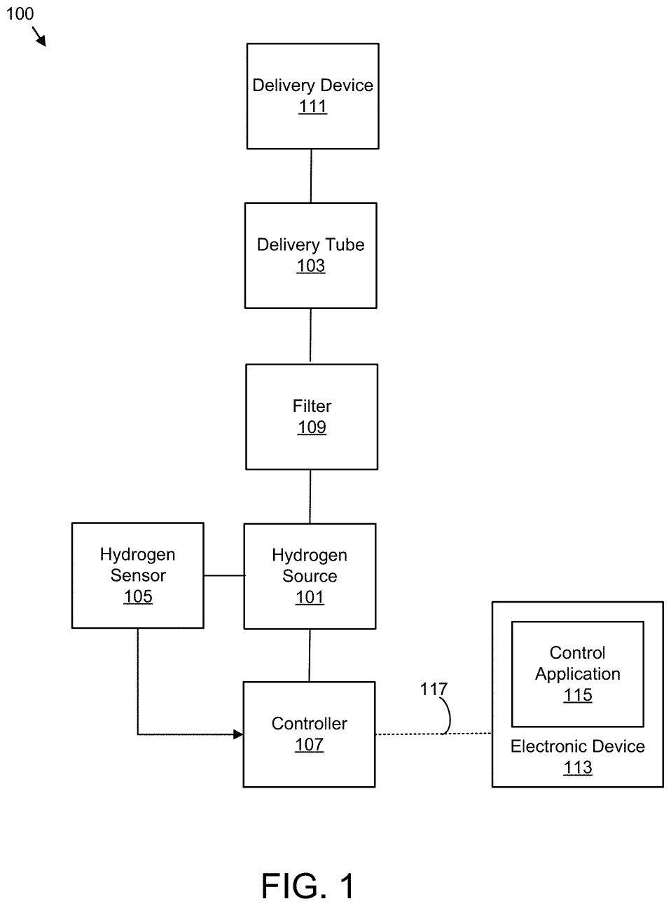

FIG. 1 is a schematic block diagram illustrating one embodiment of a hydrogen system 100. The system 100 generates and delivers hydrogen gas that neutralizes the negative effects of oxidative stress. In the depicted embodiment, the system 100 includes a hydrogen source 101 that is controlled by a controller 107. The hydrogen source 101 may be an electrolysis chamber. In addition, the hydrogen source 101 may be a hydrogen tank. The hydrogen source 101 may generate and/or provide hydrogen gas. A hydrogen sensor 105 may measure a concentration of the hydrogen gas in the hydrogen source 101. In one embodiment, the hydrogen gas is filtered by a filter 109. A delivery tube 103 may deliver hydrogen gas from the hydrogen source 101 for inhalation and/or use.

In one embodiment, the hydrogen gas is delivered via a delivery device 111. The delivery tube 103 may be connected via a unidirectional valve to a continuous positive airway pressure (CPAP) mask delivery device 111. However, some embodiments may also provide inhalation therapy through a nasal cannula or can be used to infuse water with hydrogen gas. In a certain embodiment, the delivery tube 103 is connected to a nasal cannula delivery device 111. In addition, the delivery tube 103 may deliver hydrogen to a liquid infuser delivery device 111.

The controller 107 may include Bluetooth and Wi-Fi capabilities that communicates wirelessly 117 with an electronic device 113. The electronic device 113 may run a control application 115. The control application 115 may communicate via a wireless interface to the controller 107. The control application 115 may set a concentration threshold. In addition, the control application 115 may receive a hydrogen concentration.

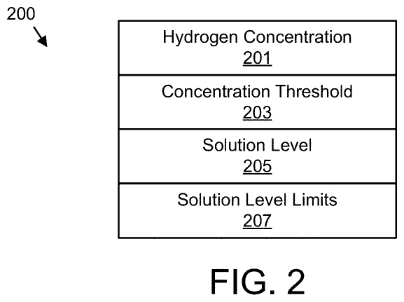

FIG. 2 is a schematic block diagram illustrating one embodiment of hydrogen data 200. The hydrogen data 200 may be used to control the generation and delivery of hydrogen gas. The hydrogen data 200 may be organized as a data structure in a memory. In the depicted embodiment, the hydrogen data 200 includes a hydrogen concentration 201, a concentration threshold 203, a solution level 205, and solution level limits 207.

The hydrogen concentration 201 may be measured by the hydrogen sensor 105 in the hydrogen source 101. In addition, the hydrogen concentration 201 may be estimated. The hydrogen concentration 201 may record the concentration of hydrogen gas in the hydrogen source 101, the filter 109, and/or the delivery tube 103.

The concentration threshold 203 may specify a maximum hydrogen concentration 201. In one embodiment, the concentration threshold 203 may be in the range of 1-4%. In a certain embodiment, the concentration threshold 203 is in the range of 1 to 3%. The concentration threshold 203 may be 1%. Alternatively, the concentration threshold may be 3%.

The solution level 205 may record a level of solution in an electrolysis chamber. The solution level limits 207 may specify a maximum and/or minimum solution level 205.

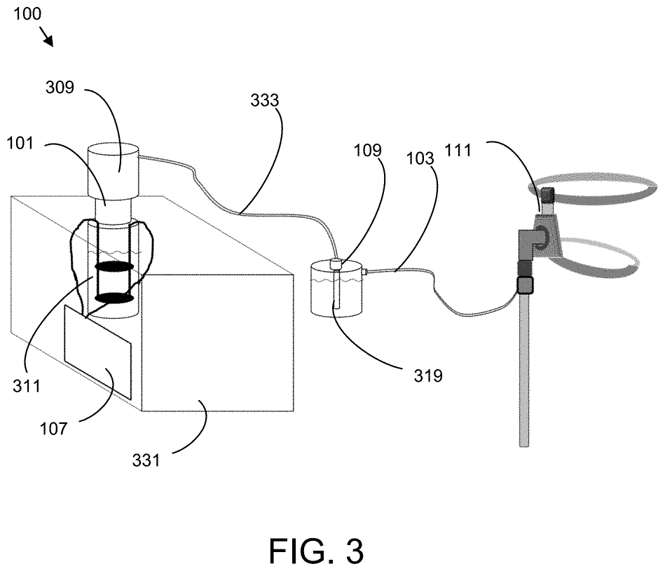

FIG. 3 is a perspective drawing illustrating one embodiment of the hydrogen system 100. The hydrogen source 101 and controller 107 are shown disposed in a housing 331. The housing 331 includes a collection cylinder 309 that connects to an electrolysis volume 311. At the top of the collection cylinder 309 there is a port for a supply tube 333 to connect the generated gas to the filter 109 as will be shown hereafter. In the depicted embodiment, the filter 109 is a water trap. The deliver tube 103 that provides hydrogen therapy to the consumer comes from the filter 109.

In the depicted embodiment, the hydrogen source 101 is an electrolysis chamber. The hydrogen source 101 generates hydrogen gas. In one embodiment, the hydrogen gas is delivered to the filter 109 via a supply tube 333. Gas enters the filter 109 through the top of the filter 109 via a trap tube 319 that descends to the bottom of the filter 109 and releases the hydrogen gas at the bottom allowing for maximum interaction with the water. Hydrogen gas emerges and leaves the filter 109 through the delivery tube 103 that connects to the delivery device 111.

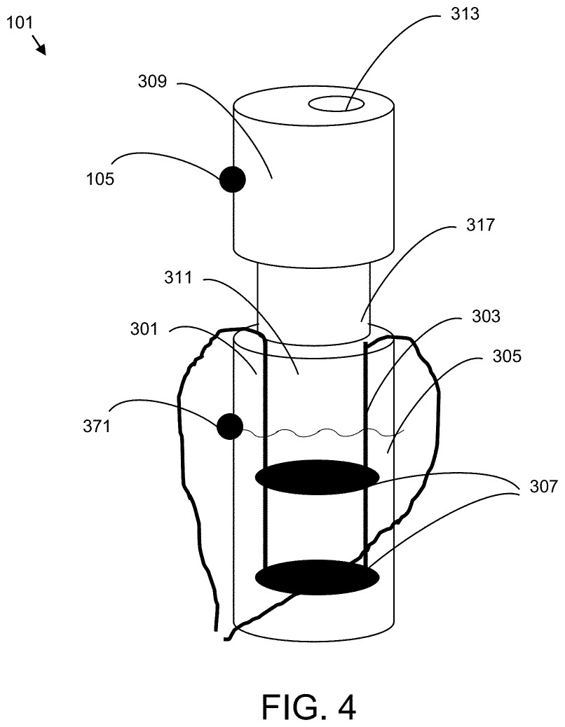

FIG. 4 is a perspective drawing illustrating one embodiment of an electrolysis chamber hydrogen source 101. The electrolysis chamber hydrogen source 101 uses the principles of electrolysis to split water molecules producing hydrogen gas. The electrolysis chamber hydrogen source 101 may be housed inside polyvinyl chloride tubing. In one embodiment, the polyvinyl chloride tubing is 4 inches in diameter and 5 inches tall. Two stainless steel electrodes 301/303 that are exposed on the top and traverse through the top of an electrolysis volume 311. Each electrode 301/303 is connected to a stainless-steel plate 307. However, none of the metal from the positive electrode 301 can be in direct contract with the metal from the negative electrode 303. The electrodes 301/303 with their plates 307 may be submerged into a lye solution consisting of sodium hydroxide (NaOH). The electrolysis chamber hydrogen source 101 may be a completely enclosed container with only a gas fitting 313 with a threaded nut on top. The electrolysis chamber hydrogen source 101 may sit flush with the ceiling of the housing 331.

The collection cylinder 309 on top of the electrolysis chamber hydrogen source 101 collects the gases generated from the electrolysis chamber 101. Oxygen may be vented. In one embodiment, the collection cylinder 309 is constructed out of PVC. A gas fitting 313 at the top of the collection cylinder 309 will allow for the connection of the supply tube 333 and/or the delivery tube 103 that will allow gas to leave and enter the delivery tube 103 and/or the filter 109.

In one embodiment, a liquid level sensor 371 in the electrolysis chamber 311 determines whether the electrodes 301/303 are submerged in solution 305, but not overfilled. A hydrogen sensor 105 may measure the hydrogen concentration 201 in the collection cylinder 309.

Details of Water Electrolysis

The electrolysis chamber hydrogen source 101 is based on the principle of water electrolysis. A positive electrode 301 and a negative electrode 303, are submerged in an aqueous solution 305. The solution 305 may be NaOH. A direct current of electricity is run through the electrodes 301/303 causing the water (H2O) of the solution 305 to split into hydrogen H₂ and oxygen O₂. The direct current electricity may be 1.23 Volts. Twice as much hydrogen gas is produced as oxygen. The gasses exit the electrolysis chamber 311 and enter a secondary water catch 317 that retains the O2, while allowing the H2 to continue through the tubing. The delivery tube 103 carrying the hydrogen gas connects to a port valve of the delivery adapter that universally fits delivery devices 111.

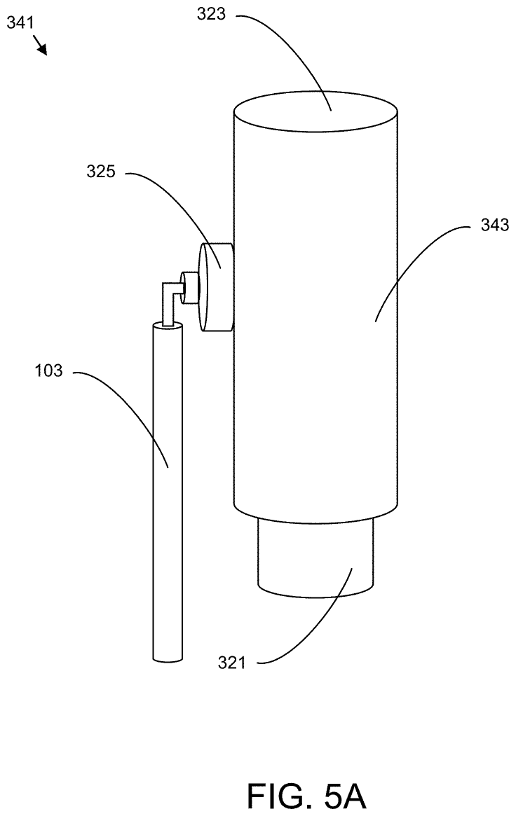

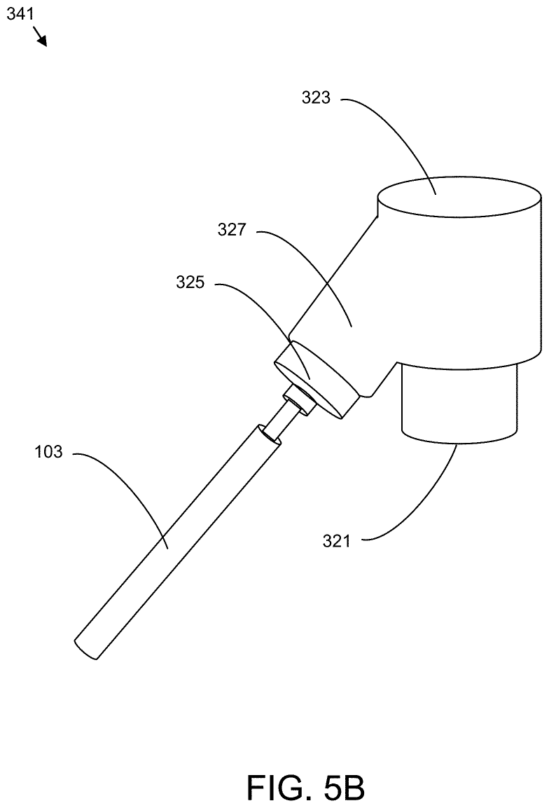

FIG. 5A is a perspective drawing illustrating one embodiment of a delivery adapter 341. The delivery adapter 341 may connect to a CPAP mask delivery device 111. The delivery adapter 341 may connect with CPAP devices. The embodiments may couple with existing CPAP machines to deliver hydrogen while one sleeps. Inhalation delivery through a delivery adapter 341 connected to a CPAP machine is the one use of the embodiments. Thus, the system 100 may be a companion device to existing CPAP machines.

Embodiments may be connected in multiple ways. One way is to connect the delivery tube 103 at the base of the tubing closest to the CPAP delivery device 111. Another way to connect the delivery tube 103 at an entry to a CPAP mask delivery device 111. The tubing for the CPAP mask delivery device 111 has rubber fittings for a female end 323 to join to the CPAP male end on the CPAP mask delivery device 111. The embodiments include an adapter tubing 343 that provides a delivery female end 323 to connect to the CPAP mask and a male end 321 to connect to the existing CPAP female end of the CPAP mask delivery device 111. In the delivery adapter 341 is a port valve 325 that allows the connection for the delivery tube 103. The port valve 325 may include a unidirectional valve that prevents air from flowing into the delivery tube 103.

The CPAP mask delivery device 111 delivers humidified air to force open breathing airways while a person sleeps. With the system 100, the CPAP mask delivery device 111 can also deliver hydrogen gas at a concentration of 10,000 parts per million (PPM) which is 20,000 times higher than the H2 gas in the ambient air that we breathe (0.5 PPM). It is also four times below the lowest level of H2 gas that can be combustible (40,000 PPM), thus maintaining safety levels. The hydrogen sensor 105 may detect leaks that would exceed 4% and would result in automatic shutoff. In a certain embodiment, the hydrogen sensor 105 is disposed in the delivery tube 103 and/or the delivery adapter 341. The hydrogen sensor 105 may measure the hydrogen concentration 201.

FIG. 5B is a perspective drawing illustrating one alternate embodiment of a delivery adapter 341. The delivery adapter 341 may connect to a CPAP mask delivery device 111. In the depicted embodiment, a bifurcating Y-shaped connection 327 includes the male end 321 and the female end 323 for connecting to the CPAP mask delivery device 111. The Y-shaped connection 327 may be made of hard plastic, but still has the male end 321 and the female end 323. The Y-shaped connection 327 may connect to the delivery tube 103 via a port valve 325. The port valve 325 may include the unidirectional valve.

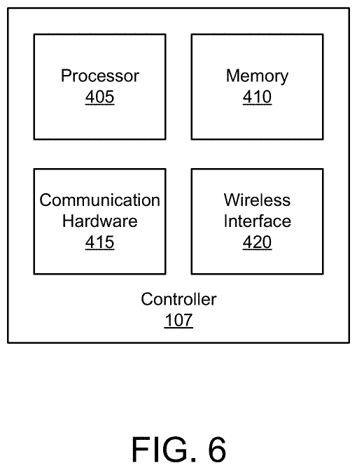

FIG. 6 is a schematic block diagram illustrating one embodiment of the controller 107. In the depicted embodiment, the controller 107 includes a processor 405, a memory 410, communication hardware 415, and a wireless interface 420. The memory 410 may store code and data. The processor 405 may execute the code and process the data. The communication hardware 415 may communicate with other elements of the system 100. The wireless interface 420 may communicate wirelessly 117 with an electronic device 113 such as a smart phone. The embodiments may include WiFi and Bluetooth capabilities in order to provide remote control if desired by the consumer.

In one embodiment, the controller 107 is a Raspberry Pi 4, but this can be modified to include a simpler and proprietary circuit board. The controller 107 controls display features on the front of the device to allow the customer to see the level of hydrogen production, the time of inhalation therapy, and the running time of the device. The controller 107 may control a fan on the back of the device to keep the electrical components cool. Additionally, the controller 107 will have the connection to the external power supply and convert that to direct current in order to provide constant voltage to the electrolysis chamber. The controller 107 have connections to sensors such as the hydrogen sensor 105 that can detect leaks of hydrogen gas that exceed the concentration threshold 203 and the liquid level sensor 371 that can detect the solution level 205 in the electrolysis chamber hydrogen source 101 to make sure the electrodes 301/303 are sufficiently covered.

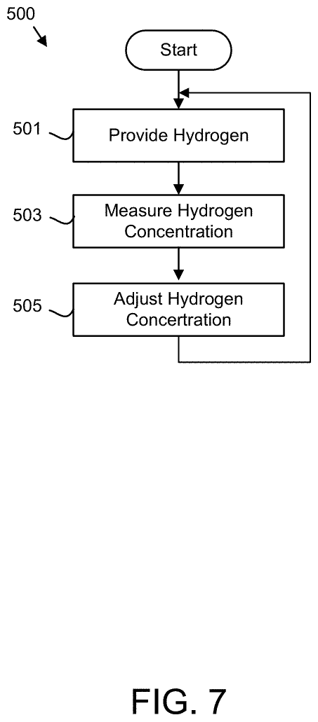

FIG. 7 is a schematic flow chart diagram illustrating one embodiment of a hydrogen gas generation and delivery method 700. The method 700 generates and delivers therapeutic hydrogen gas. The method 700 may be performed by the system 100.

The method 700 starts, and in one embodiment the method provides 501 hydrogen gas from the hydrogen source 101. The hydrogen source 101 may be controlled by the controller 107. In addition, the controller 107 may be controlled by the control application 115 and/or the electronic device 113. The hydrogen gas may be filtered by the filter 109 and delivered via the delivery tube 103.

The hydrogen sensor 105 may measure 503 the hydrogen concentration 201. The hydrogen sensor 105 may be disposed in the hydrogen source 101. In addition, the hydrogen sensor 105 may be disposed in the delivery tube 103 and/or delivery adapter 341. In one embodiment, the hydrogen sensor 105 is disposed in the electrolysis volume 311, the supply tube 333, and/or the filter 109.

The hydrogen sensor 105 may communicate the hydrogen concentration 201 to the controller 107. In one embodiment, the controller 107 communicates the hydrogen concentration 201 to the control application 115 and/or the electronic device 113.

The controller 107 may adjust 505 the hydrogen concentration 201 and the method 700 ends. The hydrogen concentration 201 may be adjusted 505 to be less than the concentration threshold 203. The controller 107 may adjust 505 the hydrogen concentration 201 using Equation 1, where h is a rate of hydrogen generation, A_{D} is a cross sectional area of the delivery device 111, A_{T} is a cross sectional area of the delivery tube 103, k₁ is a nonzero constant, and s is a control transform.

h=(A_{D}/A_{T})k₁/s Equation 1

The delivery tube 103 and delivery adapter 341 provide direct inhalation of hydrogen at the point of the delivery device 111. Additionally, the delivery tub 103 is interchangeable with a nasal cannula delivery device 111 that allows for direct inhalation without the need to connect to a CPAP mask. Lastly, the delivery tube 103 can be placed directly into water in order to generate hydrogen-infused water.

The system 100 may include one or more hydrogen sensors 105 placed within the components of the system 100 to detect any leaks that might generate greater than the concentration threshold 203 of hydrogen gas outside of the electrolysis chamber 311. This may trigger a kill switch through the controller 107 to shut off the generation of hydrogen gas. The controller 107 may include a backup battery source to prevent hard shutdowns of the system 100 so that temporary information can be saved if the wall outlet is disconnected during use.

Embodiments may be practiced in other specific forms. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus comprising:
a hydrogen source that provides hydrogen;
a delivery tube that delivers the hydrogen from the hydrogen source for inhalation;
a hydrogen sensor that measures hydrogen concentration in the delivery tube; and
a controller that adjusts the hydrogen concentration in the delivery tube to a concentration threshold.

2. The apparatus of claim 1, wherein the hydrogen source is an electrolysis chamber.

3. The apparatus of claim 1, wherein the hydrogen source is a hydrogen tank.

4. The apparatus of claim 1, wherein delivery tube is connected via a unidirectional valve to a continuous positive airway pressure (CPAP) mask delivery device.

5. The apparatus of claim 1, wherein delivery tube is connected to a nasal cannula delivery device.

6. The apparatus of claim 1, wherein delivery tube is connected to a liquid infuser delivery device.

7. The apparatus of claim 1, wherein the controller comprises a wireless interface.

8. The apparatus of claim 7, wherein a control application communicates via the wireless interface to controller, sets the concentration threshold, and receives the hydrogen concentration.

9. The apparatus of claim 1, wherein the apparatus further comprises a filter.

10. A system comprising:
a hydrogen source that provides hydrogen;
a delivery device that delivers the hydrogen;
a delivery tube that delivers the hydrogen from the hydrogen source to the delivery device;
a hydrogen sensor that measures hydrogen concentration in the delivery tube;
a controller that adjusts the hydrogen concentration in the delivery tube to a concentration threshold.

11. The system of claim 10, wherein the hydrogen source is an electrolysis chamber.

12. The system of claim 10, wherein the hydrogen source is a hydrogen tank.

13. The system of claim 10, wherein delivery tube is connected via a unidirectional valve to a continuous positive airway pressure (CPAP) mask delivery device.

14. The system of claim 10, wherein delivery tube is connected to a nasal cannula delivery device.

15. The system of claim 10, wherein delivery tube is connected to a liquid infuser delivery device.

16. The system of claim 10, wherein the controller comprises a wireless interface.

17. The system of claim 16, wherein a control application communicates via the wireless interface to controller, sets the concentration threshold, and receives the hydrogen concentration.

18. The system of claim 10, wherein the system further comprises a filter.

19. A method comprising:
providing, with a hydrogen source, hydrogen;
delivering, with a delivery tube, the hydrogen from the hydrogen source for inhalation;
measuring, with a hydrogen sensor, hydrogen concentration in the delivery tube; and
adjusting, with a controller, the hydrogen concentration in the delivery tube to a concentration threshold.
